(19) United States
(12) Patent Application Publication
(10) Pub. No.: US 2021/0315464 A1
(43) Pub. Date: Oct. 14, 2021

(54) SYSTEM FOR DETERMINING A BLOOD PRESSURE OF ONE OR A PLURALITY OF USERS

(71) Applicant: AKTIIA SA, Neuchâtel (CH)

(72) Inventors: Josep SOLÀ I CARÓS, Corcelles (CH); Mattia BERTSCHI, La Conversion (CH)

(21) Appl. No.: 17/273,618

(22) PCT Filed: Sep. 4, 2018
(86) PCT No.: PCT/IB2018/056736

§ 371 Date: Mar. 4, 2021

(51) Int. Cl. A61B 5/0205; A61B 5/00

(52) U.S. Cl. CPC A61B 5/0205; A61B 5/002; A61B 5/6824

(57) ABSTRACT

System for determining a blood pressure (BP) of users, comprising: for each user, a signal module configured to cooperate with a wearable device destined to be worn on a wrist of the user and comprising a pulsatility sensing unit; the signal module comprising a controlling module for controlling the pulsatility sensing unit to measure a plurality of pulsatility signals at the user's wrist; a processing module for processing the pulsatility signals to obtain pulsatility signal data; and a communication module for remotely transmitting said pulsatility signal data; the system further comprising an external service module including a database storage system for storing in a database the transmitted pulsatility signal data for each user; and a calculating module configured for calculating a BP value for each user, based on the pulsatility signal data stored in the database.

### FIELD

The present invention concerns a system for determining a blood pressure of one or a plurality of users.

### BACKGROUND

According to the World Health Organization, one in three adults suffer from hypertension worldwide. Hypertension can lead to severe complications, such as stroke and heart failure. Each year, this illness results in 7.5 million premature deaths worldwide. The paradox of hypertension is that most people suffering from this condition are unaware of it.

Furthermore, the current «gold standard» for blood pressure (BP) measurement is performed with a cuff placed around the arm. This 110 years old technology is cumbersome and leads to low compliance for patients prescribed to self-monitor. As a consequence, healthcare professionals lack access to complete and high-quality data for their diagnosis and the treatment of this disease.

### SUMMARY

The present disclosure concerns a non-invasive system for monitoring BP of one or a plurality of users in a continuous and accurate fashion.

More particularly, the present disclosure concerns a system for determining a blood pressure (BP) of one or a plurality of users, the system comprising:

for each user, a signal module configured to cooperate with a wearable device destined to be worn on a wrist of the user and comprising a pulsatility sensing unit; the signal module comprising a controlling module configured to control the pulsatility sensing unit such that the pulsatility sensing unit measures a plurality of pulsatility signals at the user's wrist; a processing module configured for processing the pulsatility signals to obtain pulsatility signal data; and a communication module for remotely transmitting said pulsatility signal data;

the system further comprising an external service module including a database storage system for storing in a database the transmitted pulsatility signal data for each of said one or a plurality of users; and a calculating module configured for calculating a BP value for each of said one or a plurality of users, based on the pulsatility signal data stored in the database.

In an embodiment, the pulsatility sensing unit can comprise an optical measuring sensor.

In another embodiment, the controlling module can be configured such that each of said plurality of pulsatility signals is measured during a predetermined measurement time period. The controlling module can comprise a triggering module configured to initiate and end said measurement time period.

In yet another embodiment, the pulsatility signal data corresponds to the measured pulsatility signal or the processing module can be configured to perform a pre-processing step on the measured pulsatility signals such as to obtain said pulsatility signal data.

In yet another embodiment, the signal module can cooperate with the wearable device via a first short range communication link.

In yet another embodiment, the communication module can be comprised in a portable gateway device.

In yet another embodiment, the database storage system can be configured for storing in the database the pulsatility signal data for each user.

In yet another embodiment, the system can be configured for inputting a user-specific information for each of said one or a plurality of users.

In yet another embodiment, the calculating module is configured for calculating the BP value of a user, based on the pulsatility signal data stored in the database.

In yet another embodiment, the system can comprise a display interface configured for displaying the calculated BP value.

### BRIEF DESCRIPTION OF THE DRAWINGS

The invention will be better understood with the aid of the description of an embodiment given by way of example and illustrated by the figures, in which:

FIG. 1 schematically represents a system for determining a BP of one or a plurality of users comprising a signal module, a communication module and an external service module, according to an embodiment;

FIG. 2 schematically represents details of the signal module, according to an embodiment;

FIG. 3 illustrates a possible configuration of a wearable device represents with which the signal module is destined to cooperate;

FIG. 4 shows a configuration of a pulsatility sensing unit comprising a PPG sensor, according to an embodiment;

FIG. 5 shows a schematic representation of the external service module, according to an embodiment;

FIGS. 6a and 6b respectively report exemplary pulsatility signals measured by a pulsatility sensing unit on a wearable device as a function of time;

FIG. 7a reports an exemplary activity level of a user as a function of time and FIG. 7b shows a pulsatility signal measurement being triggered after a predetermined resting time period;

FIG. 8a reports another exemplary activity level of a user as a function of time, FIG. 8b shows a detected “intense” exercise time period and FIG. 8c shows a pulsatility signal measurement being triggered after a predetermined resting time period;

FIG. 9 shows a flowchart representing a decision tree for triggering the measurement of the pulsatility signal, according to an embodiment;

FIG. 10 illustrates an arrangement wherein the signal module is completely comprised in the wearable device, according to an embodiment;

FIG. 11 illustrates an arrangement wherein at least a part of the signal module is comprised in the wearable device, according to an embodiment;

FIG. 12 represents the system comprising a portable gateway device, according to an embodiment;

FIG. 13 shows the system comprising a portable gateway device, according to another embodiment;

FIG. 14 shows the system comprising a portable gateway device, according to yet another embodiment;

FIG. 15 shows a database of the external service module, according to an embodiment; and

FIG. 16 shows a database of the external service module, according to another embodiment.

### DETAILED DESCRIPTION OF POSSIBLE EMBODIMENTS

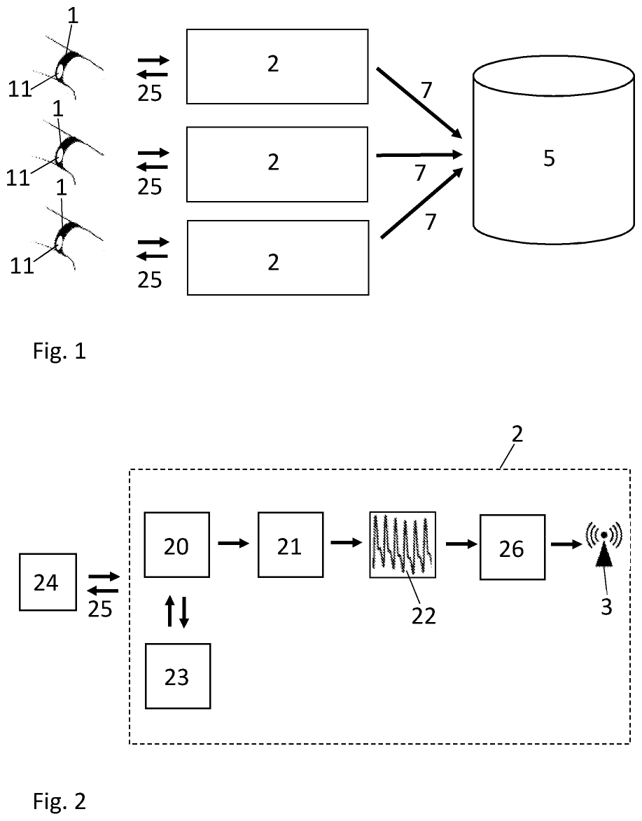

FIG. 1 schematically represents a system for determining a blood pressure (BP) value of one or a plurality of users, according to an embodiment. For each user, the system comprises signal module 2 cooperating with a wearable device 1 destined to be worn on a wrist of the user and comprising a pulsatility sensing unit 11. The pulsatility sensing unit 11 can comprise an analog-to-digital converter module (not shown) outputting pulsatility signals 4.

FIG. 2 represents a possible embodiment of the signal module 2. The signal module 2 includes a controlling module 20 configured to control the wearable device 1 such that the pulsatility sensing unit 11 measures pulsatility signals 4 at the user's wrist when the wearable device 1 is worn. The signal module 2 further comprises a processing module 21 configured for processing the pulsatility signals such as to obtain pulsatility signal data 22.

Wearable Device

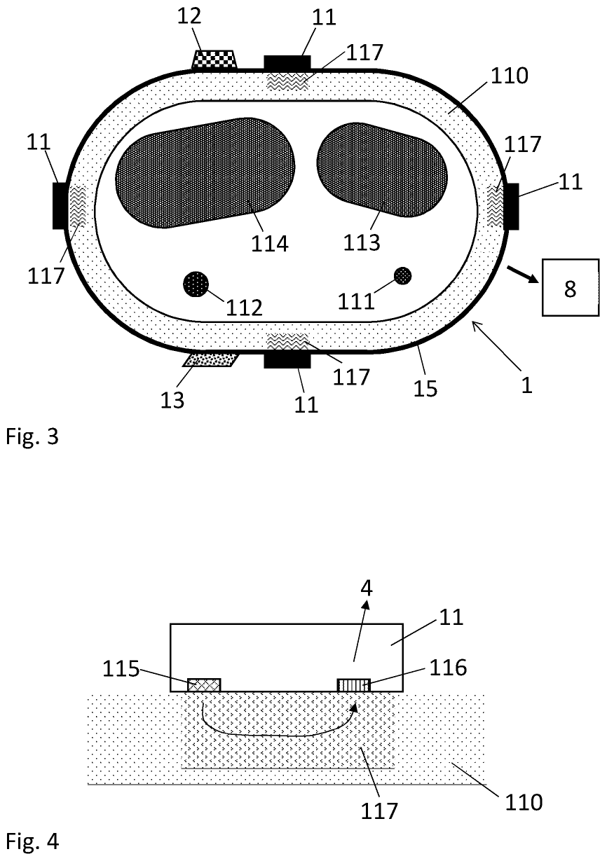

A possible configuration of the wearable device 1 is illustrated in the cross section view of FIG. 3. The wearable device 1 may include a wristband 15 containing the pulsatility sensing unit 11. In the specific example of FIG. 3, the wristband 15 comprises four pulsatility sensing unit 11 distributed along the inner side of the wristband 15 periphery such as to be in contact with the user's wrist skin when the wearable device 1 is worn. Other arrangements of the pulsatility sensing unit 11 on the armband are possible. For example, the armband can comprise any number of pulsatility sensing unit 11 in various configurations along the wristband 15.

In one embodiment, the pulsatility sensing unit 11 may comprise a photoplethysmograph (PPG) sensor array that may measure arterial pulsation, arterial diameter, blood flow and/or blood content. In this embodiment, the pulsatility sensing unit 11 may be arranged on the wristband 15 so that the optical sensor array 11 straddles or otherwise addresses an artery, such as the ulnar artery 111, in the vicinity of the ulna bone 113, or radial artery 112, in the vicinity of the radius bone 114 (as shown in FIG. 3) or any arterial vascular bed 117 of the skin of the wrist.

A detail of a possible configuration of the PPG sensor 11 is shown in FIG. 4. The PPG sensor 11 comprises at least one light sources 115 and at least one photodetector 116 located adjacent to the light source 115. For example, the PPG sensor 11 can comprise two or more light sources emitting at same wavelength or different wavelengths. The light source 115 may comprise light emitting diode (LED). The light source 115 may further comprise any other appropriate source such as a laser, incandescent or ambient light. The photodetectors 116 may comprise phototransistors, a camera imaging device or charge-coupled devices (CCD). The pulsatility sensing unit 11 may comprise any other suitable optical sensor such as at least one of: a laser speckle sensor, a laser Doppler sensor, or a camera.

The pulsatility signal 4 measured by the PPG sensor 11 can be defined as a signal containing information on the periodic variation of blood flow and arterial diameter of a given segment of the arterial tree. The periodic variations are typically generated by the arrival of a pressure pulse at the given segment of the arterial tree. In the configuration of FIGS. 3 and 4, the pulsatility signal 4 corresponds to a reflective photoplethysmograph signals wherein the light emitted by the light source 115 passes through the capillary bed 117 of the user's wrist skin. Other arrangements of the PPG sensor 11 are possible in order to measure a transmittance photoplethysmograph signals.

In other arrangements, the wearable device 1 may comprise a strip of material that is to be worn on another body part of the user. Examples of the wearable device 1 may include, but are not limited to an armband, a headband, an ankle bracelet, a choker, and a ring, a helmet, an ear plug, a hearing aid, a headphone, glasses, a shirt, a bra, a garment, a fingertip sensor, a glove, underpants, a socket, a shoe, a wearable sensor, a patch adhering to the skin of the user, a bed sensor, a chair sensor, a toilet sensor, a table sensor, a car sensor, a computer mouse sensor, or any other arrangement intended to measure a pulsatility signal.

For example, the wearable device 1 may include any existing device including an armband device, a smartwatch or any or device worn at a user's wrist and comprising the pulsatility sensing unit 11 adapted to measure pulsatility signals at the user's wrist.

In other arrangements, the wearable device 1 may include, in addition to, or instead of the pulsatility sensing unit 11, other type of sensors, such as a galvanic skin response (GSR) sensor array, a bioimpedance (BioZ) sensor array, an electrocardiography sensor (ECG), a sensor based on radio frequency (RF) detection, a radar sensor, a mechanical sensor, a pressure sensor, an invasive sensor, an intra-arterial sensor, a minimal invasive sensor, a subcutaneous sensor, a tonometer, a strain sensor, a plethysmographic sensor, a microphone, an ultrasound sensor, a capacitive sensor, an electromagnetic sensor, a Raman sensor, or any sensor capable of measuring a pulsatility signal either from the capillary bed of the skin or from any other section of the arterial tree.

Turning back to FIG. 1, the system comprises an external service module 5 remote from the wearable devices 1 and from the signal modules 2, and configured for receiving the pulsatility signal data 22 from the signal modules 2.

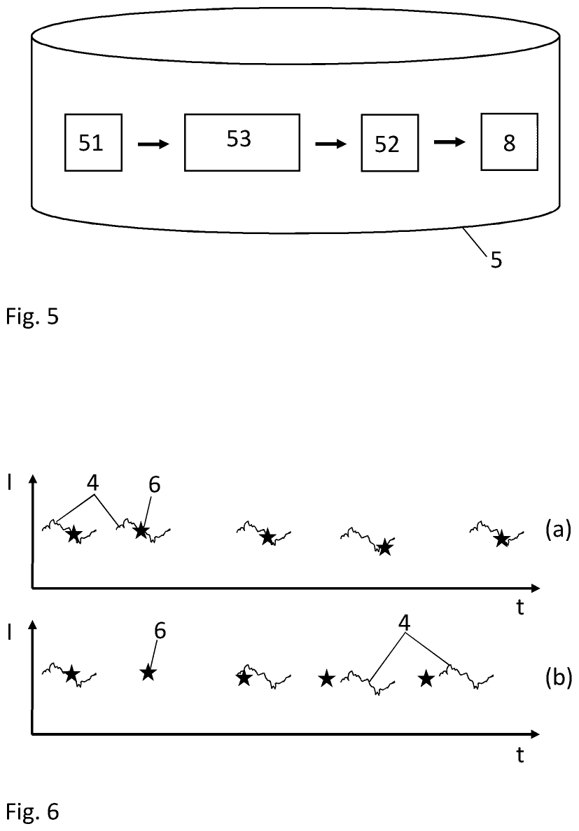

A schematic representation of the external service module 5 is shown in FIG. 5, according to an embodiment. The external service module 5 comprises a database storage system 51 configured for storing the received pulsatility signal data 22 in a database 53. The external service module 5 further comprises a calculating module 52 configured for calculating a BP value for each user, based the pulsatility signal data 22 stored in the database 53.

The external service module 5 is typically remote from the signal modules 2. In an embodiment, a communication module 3 is used for transmitting the pulsatility signal data from the signal module 2 to the external service module 5. The external service module 5 can comprise one or a plurality of remote servers (or computers). The one or a plurality of remote servers can be in a single location or the plurality of remote servers can be geographically distributed (such as in a computer network or cloud computing). The database storage system 51, the calculating module 52 and/or the database 53 can be distributed across the plurality of remote servers.

As shown in FIG. 1, a plurality of signal modules 2, each cooperating with a wearable device 1, can transmit their pulsatility signal data 22 to the external service module 5. The database storage system 51 can then store a set comprising a plurality of pulsatility signal data 22 obtained for each wearable device 1 (for each user).

Measuring-Triggering

In an embodiment, the controlling module 20 can be configured such that each of said plurality of pulsatility signals is measured during a predetermined measurement time period.

FIGS. 6a and 6b respectively report exemplary pulsatility signals 4 measured by the pulsatility sensing unit 11 on a wearable device 1 as a function of time. The scale on the ordinate corresponds to an arbitrary intensity unit. The pulsatility signals 4 can correspond to several measurements performed on a user during a measurement time period. The pulsatility signals 4 can be measured at regular or irregular. The measurement time period can be the same from a pulsatility signal 4 to another or can differ.

In an embodiment, the controlling module 20 comprises a triggering module 23 (see FIG. 2) configured to initiate or stop measuring pulsatility signals 4 by the pulsatility sensing unit 11.

The triggering module 23 can control the pulsatility sensing unit 11 according to a trigger parameter. The trigger parameter can be specific to a user.

Examples of trigger parameter can include a trigger signal such as a motion signal representative a user's movement. Such motion signal can be measured by using a motion sensor 12 placed on the user, for example on the wearable device 1. The motion signal can be used for calculating an activity level of the user and the activity level compared to a threshold value. The triggering module 23 can control the pulsatility sensing unit 11 such that a pulsatility signal measurement is initiated or stopped when the activity level is above or below the threshold value.

The motion signal and/or the pulsatility signal 4 can be used for detecting that the wearable device is worn by the user.

The motion sensor 12 can include any one of an inertial measurement unit (IMU), an accelerometer, a gyroscope, magnetometer, or a combination of these devices.

In another embodiment, the trigger parameter includes a trigger signal such as a geolocation information of the user. The geolocation information can be provided by a geolocation sensor 13 (such as a GPS device) worn by the user, for example, a geolocation sensor 13 comprised on the wearable device 1. The geolocation information can be used, for example, to allow measuring with the pulsatility sensing unit 11 when the user is in a given area such as at home, in the working place, and prevent measurements otherwise. The geolocation information can also be provided by any indoor positioning system that is interfaced with the geolocation sensor 13 (such as nodes from a WiFi/LiFi access points, Bluetooth beacons, or any other optical, radio or acoustic localization technology) also worn by the user. The geolocation information can also be provided by any other positioning system integrated in the geolocation sensor 13 (such as magnetic or inertial positioning systems) also worn by the user. The geolocation information can be used, for example, to allow measuring with the pulsatility sensing unit 11 when the user is in a given area such as at home, in the working place, at a certain living environments, and promote or prevent measurements accordingly.

In yet another embodiment, the trigger parameter comprises a trigger signal such as a behavioral information on the user. Behavioral information can comprise: known awake/asleep patterns of the user, predetermined measurement schedule, or specific measurement schedule. Known awake/asleep patterns or active/sedentary patterns can be manually introduced in the system (in the database 53) or learnt by the system. Predetermined measurement schedule can be introduced by a clinician, for instance depending on a drug therapy schedule or depending on the needs of a particular investigation to be performed on the user. The schedule can be fixed for a specific user, or dynamically modified for the investigation needs. Specific measurement schedule can comprise a measurement schedule manually introduced depending on working, home, leisure plans of the user, geo-localization of the user or new events occurring in the user life.

Behavioral information can further comprise a feature connected with the user agenda that automatically adapts the measuring frequency according to the scheduled activities.

In yet another embodiment, the trigger parameter can comprise an activity level, determined from the motion signal, and/or an exercise detection. Here, the trigger parameter can be used in combination with a predetermined time period during which the user is at rest after the activity and/or exercise.

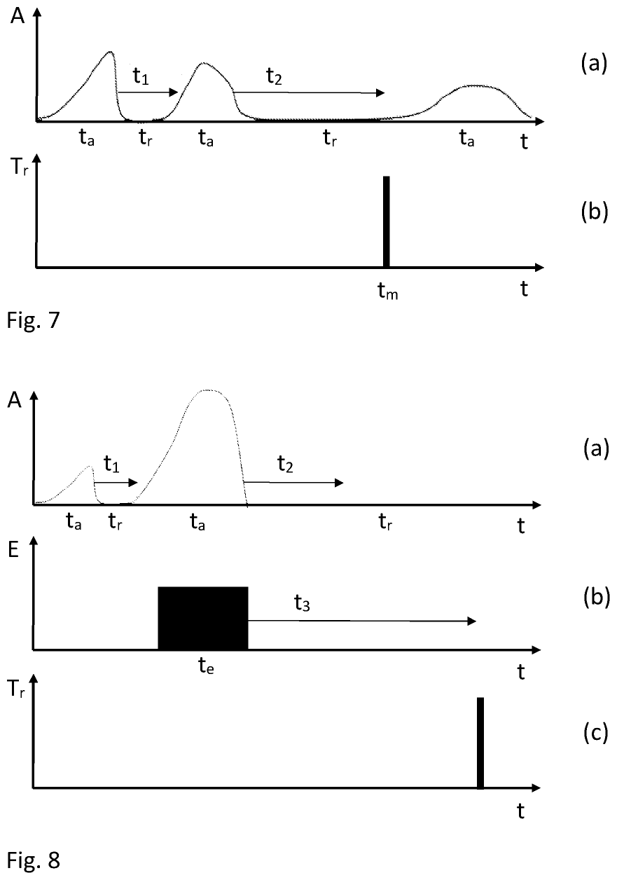

FIG. 7a reports an exemplary activity level of a user calculated from the motion signal as a function of the elapsed time. In particular, FIG. 7a shows active time periods to where the user is active and resting time periods tᵣ where the user is at rest. FIG. 7b shows that the triggering module 23 triggers (initiates) a measuring pulsatility signal 4 by the pulsatility sensing unit 11 at a triggering time tₘ, once the duration of a resting time periods tᵣ is more than a predetermined resting period after activity (second resting period t₂). The duration of the first resting period t₁ is less than the predetermined resting period after activity and no pulsatility signal measurement is initiated.

FIG. 8a reports another exemplary activity level of a user calculated from the motion signal as a function of the elapsed time. FIG. 8b reports a detected “intense” exercise time period tₑ possibly calculated from the motion signal, and/or the geolocation sensor 13, and corresponding to the user performing an activity with an intensity being above a given intensity level. FIG. 8c shows that the pulsatility signal measurement is triggered only when the two following criteria are satisfied: the duration of a resting time period tᵣ is more than the predetermined resting period after activity (second resting period t₂) and more than a predetermined resting period after “intense” exercise (third resting period t₃).

Pulsatility signal measurements performed during “intense” exercise, during activity and/or during any type of mental or cardiovascular stresses are biased and might lead to wrong clinical interpretations. The trigger parameters can thus be used for detecting such stresses, and further used for tracking elapsed time after the end of each of them. The system can then trigger the initiation of a measurement when the elapsed time are above predetermined thresholds. For example, according to guidelines for measuring/monitoring blood pressure (such as “Home Blood Pressure Monitoring Explained” by the British Hypertension Society), the predetermined resting period after activity can be about 5 min and the predetermined resting period after “intense” exercise can be about 30 min.

Indeed, blood pressure measurements should be performed after the user has been resting for at least 5 min, and not having exercised for at least 30 min. Measurements performed during exercise and/or during any type of mental or cardiovascular stresses are biased and may lead to wrong interpretations. The trigger parameters can thus be used for detecting such stresses, and further used for tracking one or several elapsed time periods after the end of each of any type of activity or stress.

In yet another embodiment, the trigger parameter can comprise the activity level and/or an exercise detection in combination with at least another trigger parameter, such as behavioral information, geolocation information, etc. In fact, the trigger parameter can correspond to any criteria define in the guidelines. The triggering module 23 can use the different trigger parameters in a hierarchical fashion, or in a decision tree manner, in order to initiate the pulsatility signal measurement.

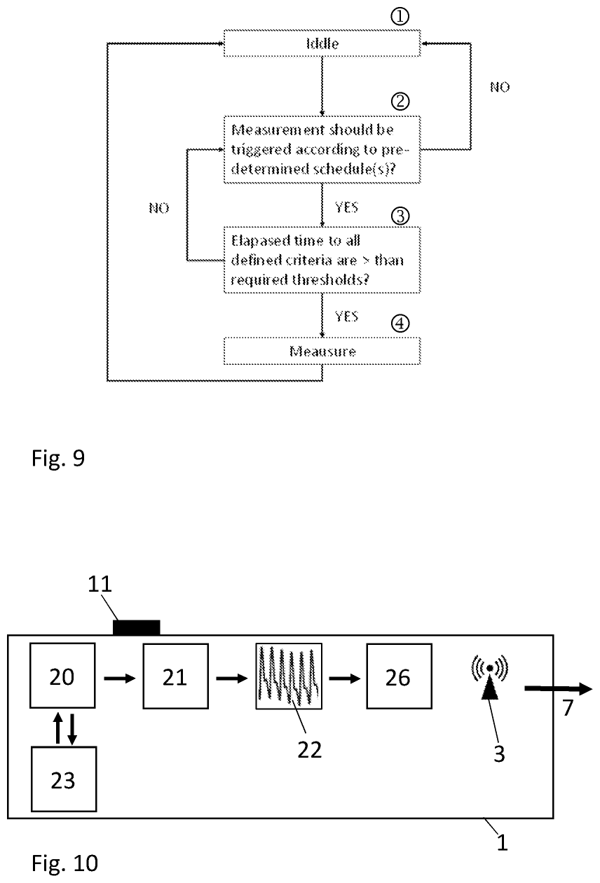

FIG. 9 shows a flowchart representing a possible decision tree algorithm for triggering the pulsatility signal measurement using at least two different triggering parameters. The triggering module 23 is idle (step \textcircled{1}). The triggering module 23 then check if a triggering parameter comprising a behavioral information, for example if a criterion corresponding to a predetermined measurement schedule, is fulfilled (step \textcircled{2}). The triggering module 23 then check if a condition set by a triggering parameter comprising an activity level and/or exercise detection in combination with a predetermined resting period, is fulfilled (step \textcircled{3}). Once the conditions of steps \textcircled{2} and \textcircled{3} are fulfilled, the measurement is initiated (step \textcircled{4}).

In an embodiment, the trigger parameter can be transmitted to the external service module 5 via the communication module 3. The storage device 51 can then be configured to store the transmitted trigger parameter in the database 53.

In yet another embodiment, the triggering module 23 control the pulsatility sensing unit 11 according to a manual input. The communication module 3 can be configured for transmitting the triggering input to the external service module 5.

Pre-Processing

In an embodiment, the processing module 21 can be configured such that the measured pulsatility signals 4 are transmitted to the external service module 5 and stored in the database 53. In other words, no processing is performed on the “raw” pulsatility signals 4.

In an embodiment, the processing module 21 can be configured to perform a pre-processing step on the measured pulsatility signals 4, such that the pulsatility signal data 22 correspond to measured pulsatility signals 4 having been submitted to the pre-process step.

In a variant, the processing module 21 is configured to perform a pre-processing step on the measured pulsatility signals 4 such as to obtain said pulsatility signal data 22.

The pre-processing step comprises a lossless compression of the measured pulsatility signal 4. Examples of such lossless compression is described in the reference: J. Uthayakumar et. al., “A survey on data compression techniques: From the perspective of data quality, coding schemes, data type and applications”, available online 17 May 2018, https://doi.orq/10.1016/j.jksuci.2018.05.006.

Alternatively, the pre-processing step comprises a lossy compression of the measured pulsatility signal 4. An example of such lossless compression is described in the same reference.

Alternatively, the pre-processing step comprises executing an ensemble averaging algorithm on the measured pulsatility signal 4.

A possible ensemble averaging algorithm can comprise the steps of:

identifying individual arterial pulses in a sequence of optical signals (by instance by detecting a local maxima or a local minima of the signals);

selecting a window of data around each identified pulse (by instance, a 1 sec window length);

weighting each individual pulse according to a reliability criteria (by instance, setting to “0” too noisy pulses;

overlapping the identified pulses, weighted by the respective weighing factor; and

estimating the most likely ensemble average representing all the pulses in the sequence of optical signals (by instance, by performing the arithmetic average of the weighted pulses).

Buffering

In an embodiment, the signal module 2 is remote from the wearable device 1. The signal module 2 can communicate with the wearable device 1 via a first short range communication link 25 (see FIG. 1).

The first short range communication link 25 can include an optical or radio wave communication device, notably using RFID and near-field communication, the Bluetooth® transmission protocol, Bluetooth Low Energy (BLE), a near field communication protocol (NFC), a proximity card or a WiFi direct connection, Zigbee, power line communication, infrared transmission (IR), ultrasound communication, a Z-wave protocol or any other home automation communication protocol.

The short range communication link 25 can comprise a first short range data buffer 24 (see FIG. 2) adapted to store the measured pulsatility signals 4 when they are not transmitted by first short range communication link 25. For example, the first short range data buffer 24 can store the measured pulsatility signals 4 when the wearable device 1 and/or the signal module 2 are out of range or any one of them is disabled, or the short range communication link 25 is not available or not working.

The communication module 3 can be configured for remotely transmitting the pulsatility signal data 22 from the signal module 2 to the external service module 5 via a long range communication link 7 (see FIG. 1). The long range communication link 7 can include a mobile telephone network or a computer networks using the Internet Protocol.

The signal module 2 can comprise a long range data buffer 26 adapted to store the pulsatility signal data 22 when they are not transmitted by communication module 3 to the external service module 5. Again, the long range data buffer 26 can store the pulsatility signal data 22 when the signal module 2 and/or the external service module 5 are out of range or any one of them is disabled, or the long range communication link 7 is not available or not working.

The first short range data buffer 24 and long range data buffer 26 can comprise a region of a physical memory storage provided in the signal module 2 used to temporarily store, respectively, the measured pulsatility signals 4 and the pulsatility signal data 22 while not transmitted by the short range communication link 25 and the communication module 3, respectively. The first short range and long range data buffer 24, 26 can be implemented in a fixed memory location in hardware or by using a virtual data buffer in software, pointing at a location in the physical memory.

Other Arrangements of the Wearable Device and the Signal Module

In an embodiment illustrated in FIG. 10, the signal module 2 is completely comprised in the wearable device 1. In the example shown, the controlling module 20, processing module 21, triggering module 23, the long range data buffer 26 and the communication module 3. In this configuration, there is no need for the short range communication link 25 (and for the first short range data buffer 24) since the pulsatility signal data 22 are transmitted directly from the wearable device 1, via the long range communication link 7 by the communication module 3, toward the external service module 5.

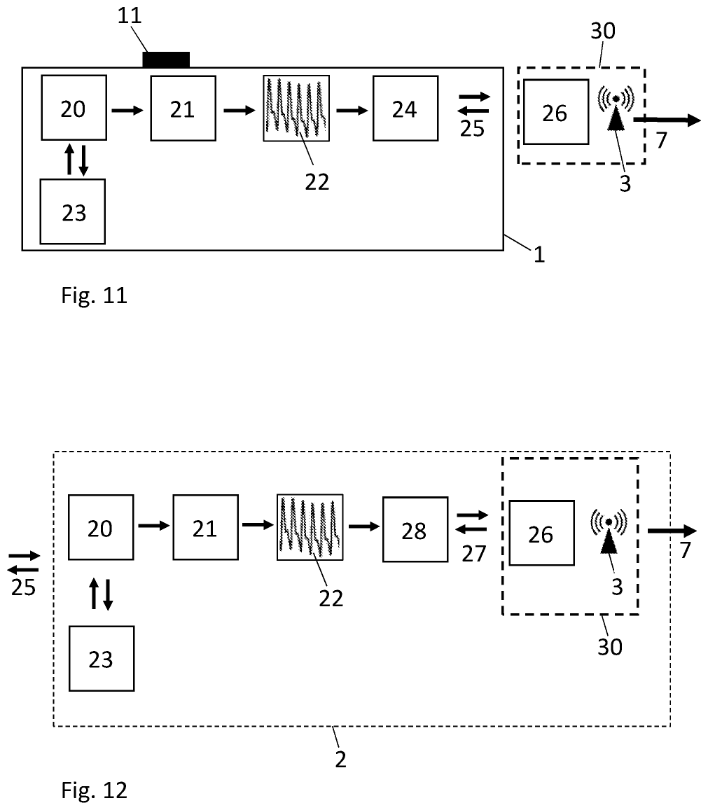

In another embodiment illustrated in FIG. 11, at least a part of the signal module 2 is comprised in the wearable device 1, the other part being remote form the wearable device 1. In the example, the controlling module 20, processing module 21, triggering module 23 and the short range communication link 25 (and possibly the first short range data buffer 24) are comprised in the wearable device 1. The communication module 3 and possibly the long range data buffer 26 are remote from the wearable device 1. The pulsatility signal data 22 are transmitted from the wearable device 1, via the short range communication link 25 to the communication module 3.

The controlling module 20 can comprise a controlling firmware portion that can be included in the wearable device 1 and configured to cooperate with the wearable device 1.

The controlling module 20 can further comprise a controlling hardware portion that can also be included in the wearable device 1.

In a further possible embodiment, the processing module 21 can comprise a processing firmware portion that can be included in the wearable device 1.

In a further possible embodiment, the triggering module 23 can comprise a processing firmware portion that can be included in the wearable device 1.

It should be understood that the firmware portion may also comprise a middleware, a software portion or any executable code.

The first short range communication link 25 can be placed between the part of the signal module 2 being comprised in the wearable device 1 and the part being remote form the wearable device 1. The first short range data buffer 24 can then be between the part of the signal module 2 being comprised in the wearable device 1 and the first short range communication link 25. In the example of FIG. 11, the first short range data buffer 24 and the first short range communication link 25 are between the processing module 21 and the communication module 3 (the latter being remote from the wearable device 1).

Portable Gateway Device

In an embodiment, the signal module 2 can be comprised at least in part in a portable gateway device 30. The portable gateway device 30 can comprise an electronic mobile device such as a smartphone, a tablet, a laptop, a desk computer or any other suitable device capable of communicating with the signal module 2 and the external service module 5.

In the embodiment of FIG. 11, the communication module 3, possibly with the long range data buffer 26, is comprised in a portable gateway device 30 while the rest of the signal module 2 is comprised in the wearable device 1. In such configuration, the communication module 3 can be part of the portable gateway device 30 such as the antenna in a smartphone. The first short range communication link 25 can be comprised in the wearable device 1 and communicate with the portable gateway device 30.

The portable gateway device 30 can be configured for remotely transmitting said pulsatility signal data 22 via the long range communication link 7.

In another embodiment shown in FIG. 12, the signal module 2 is remote from the wearable device 1 (such as in FIG. 1) and the communication module 3 is comprised in the portable gateway device 30. The signal module 2 can comprise a second short range communication link 27 for transmitting the processed data 22 to the portable gateway device 30. A second short range data buffer 28 can then be placed before the second short range communication link 27 to store the pulsatility signal data 22 when they are not transmitted by second short range communication link 27.

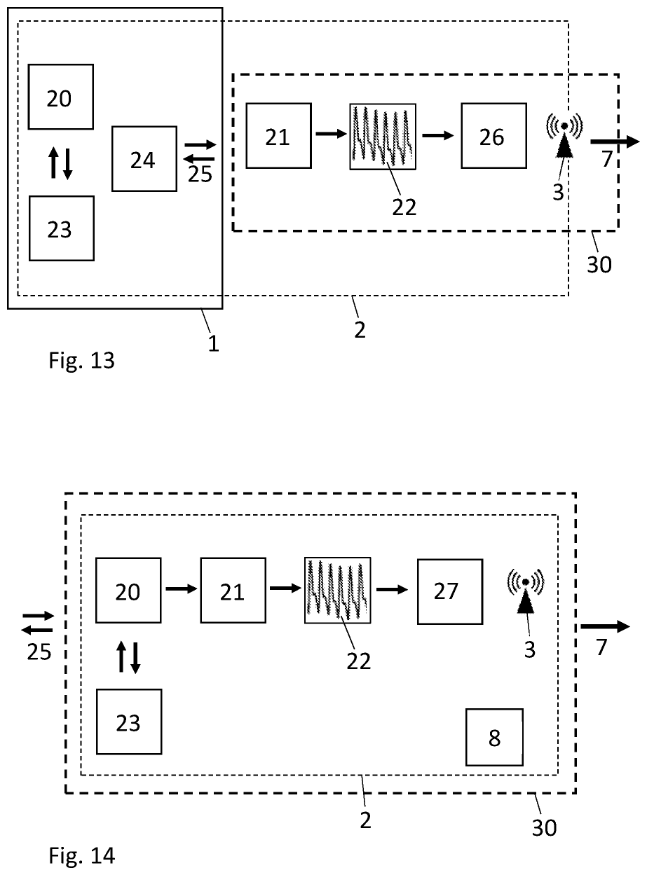

In another embodiment shown in FIG. 13, the controlling module 20, the triggering module 23 and the first short range communication link 25 (possibly with the first short range data buffer 24) are comprised in the wearable device 1 while the remote portable gateway device 30 comprises the processing module 21 and the communication module 3. The portable gateway device 30 can further comprises the long range data buffer 26 before the communication module 3.

In a further variant shown in FIG. 14, the signal module 2 (comprising the communication module 3) can be completely comprised in the portable gateway device 30. In this configuration, the portable gateway device 30 can communicate with the wearable device 1 via the first short range communication link 25 and with the external service module 5 via the long range communication link 7 provided by the communication module 3. In this configuration, there is no need for the second short range communication link 27 and second short range data buffer 28.

The portable gateway device 30 can be advantageously used as an inputting means.

For example, the triggering input can be entered via the portable gateway device 30.

In a possible configuration, the motion sensor 12 can be comprised in the portable gateway device 30. For example, the accelerometer or geolocation means in a smartphone can be used for this purpose. Alternatively, the motion sensor 12 can be comprised in any other device such as a commercial fitness tracker.

Database

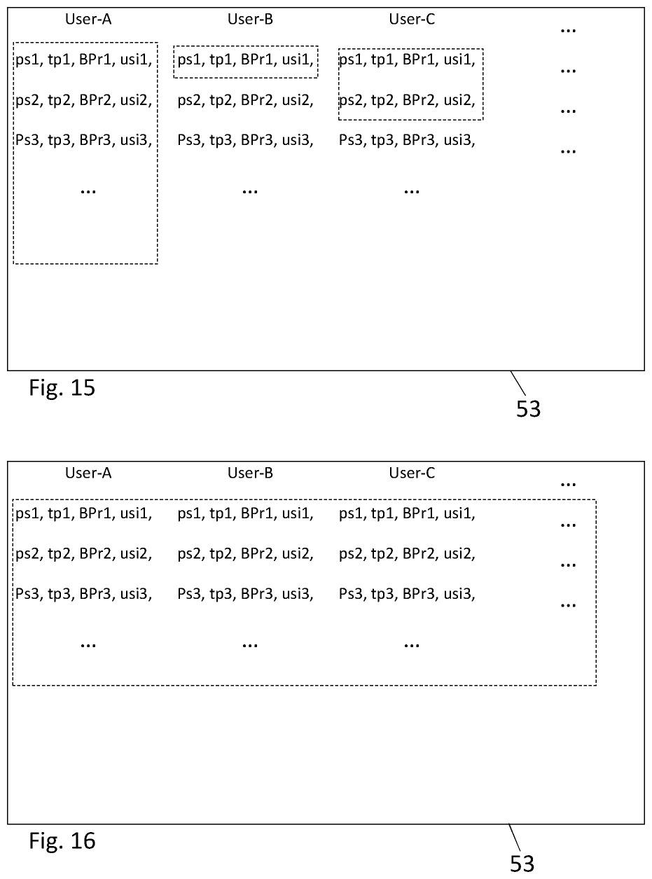

The database storage system 51 is configured for storing the pulsatility signal data 22 in a database 53. FIG. 15 shows a database 53 of the database storage system 51, according to an embodiment.

Turning back to FIG. 1, a plurality of signal modules 2, each cooperating with a wearable device 1, can transmit their pulsatility signal data 22 to the external service module 5. The database storage system 51 can then store a set comprising a plurality of pulsatility signal data 22 obtained for each wearable device 1 (for each user).

In the particular example of FIG. 15, the database 53 stores a set comprising a plurality of pulsatility signal data 22 obtained for a user. For example, for the user A, the database 53 stores the set of pulsatility signal data 22 (ps1, ps2, ps3, . . . or psi with i=1 to n, where n is the number of measurements). For the user B, the database 53 stores another set of pulsatility signal data 22 (psi with i=1 to n), etc.

The database 53 can further store trigger parameters (indicated by the symbol “tpi” with i=1 to n) corresponding to each user and to each set of pulsatility signal data 22.

The database storage system 51 can be further configured for storing the triggering input in the database 53 (not shown) for each user and set of pulsatility signal data 22.

The database storage system 51 can be configured such that a set of pulsatility signal data 22 corresponding to a user is stored in the database 53 separately from another set of pulsatility signal data 22 corresponding to another user.

User-Specific Information

In an embodiment, the system is configured for inputting a user-specific information for each of said one or a plurality of users.

The user-specific information can comprise any one of: type of wearable device worn by the user, age, weight, ethnics information, gender, skin color, cardiovascular condition, information related to the user's, mood, intake of food and drinks, type of activity performed by the user, drug intakes and dosages, weather, physical condition, alcohol consumption, smoking history, known health issues, health records or a combination thereof.

The user-specific information can further comprise one or a plurality of reference BP measurements. The reference BP measurements can be performed independently from to the measurement made with the pulsatility sensing unit 11.

Independent BP measurement can comprise measurements performed using any appropriate non-invasive measurement technique, including manual measurement by a health care professional, automatic measurement by an automated brachial cuff, automatic measurement by an automated wrist cuff or any appropriate invasive measurement technique, including an invasive arterial line.

In an embodiment illustrated in FIG. 6a, one or a plurality of reference BP measurements 6 is measured simultaneously with the measurement of the pulsatility signal 4.

In another embodiment illustrated in FIG. 6b, one or a plurality of reference BP measurements 6 is measured independently from the measurement of the pulsatility signal 4.

In yet another embodiment, the communication module 3 is configured for transmitting said user-specific information to the external service module 5. The database storage system 51 can then be configured such that user-specific information corresponding to each set of pulsatility signal data 22 is stored in the database 53. In particular, user-specific information corresponding to each pulsatility signal data 22 in a set of pulsatility signal data 22 (indicated by the symbol “usi” with i=1 to n) can be stored in the database 53.

Calculating

The external service module 5 further comprises a calculating module 52 configured for calculating a BP value of a user, based the pulsatility signal data 22 stored in the database 53.

The external calculating module 52 can be further configured for calculating a quality index value of the pulsatility signal of the user, based on the pulsatility signal data 22 stored in the database 53. The quality index value of a pulsatility signal can be calculated from the pulsatility signal 4 and can be used for quantifying the quality of the measured signal 4. The quality index value can be calculated from: the signal to noise ratio of the pulsatility signal, the likelihood of the pulsatility signal 4 able to be analyzed by the calculating module 52, the presence of physiological features within the pulsatility signal 4 (for instance, the physiological features described in European patent application EP3226758), or any combination thereof. The quality signal value can also be stored in the database 53 as a user-specific information.

In an embodiment, the calculating module 52 can be configured for calculating the BP value of a user based on the set comprising a plurality of pulsatility signal data 22 obtained for the user.

In a variant, the calculating module 52 can be configured for calculating the BP value of a user, based on a subset of the set comprising a plurality of pulsatility signal data 22. In the example of FIG. 15, the BP value of user A is calculated by using the set comprising a plurality of pulsatility signal data 22 obtained for this user (shown by the dotted square). In the case of user C, the BP value is calculated by using a subset comprising two pulsatility signal data 22 obtained for that user.

In a further embodiment, the calculating module 52 can be configured for the BP value of a user using a subset comprising only one pulsatility signal data 22 (see FIG. 15, user B). In such configuration, the BP value can be calculated for each measured pulsatility signal.

In another embodiment shown in FIG. 16, the calculating module 52 can be configured for calculating the BP value of a selected user, based on the pulsatility signal data 22 stored in the database 53 from a subset of users, a subset of user parameters and/or user specific information (trigger parameter). Calculating the BP value using the pulsatility signal data 22 obtained from the subset of users, possibly in combination with the subset of user parameters and/or user specific information parameters, allows for improving the performances of the calculation module 52 by providing more data for training the calculating technique. The subset of users can be clustered according to the user-specific information (users having the same skin color, etc.) or according to trigger parameter (user for which the pulsatility signal measurements were triggered the same way).

The subset of users can further be clustered by using the clustering procedure disclosed in patent application US20130041268, or any other clustering or classification technique. As clustering space, any combination of the following features can be used: the signal to noise ratio of the pulsatility signal data, the likelihood of the pulsatility signal data 22 able to be analyzed by the calculating module 52, the presence of physiological features within the pulsatility signal (for instance, the physiological features described in European patent application EP3226758), or any other feature that can be calculated from the pulsatility signal data 22. Pulsatility signal data 22 can be automatically clustered from different users.

The clustered pulsatility signal data 22 can then be used altogether to better train the algorithms for those users. The clustered pulsatility signal data 22 are independent of from specific user or of the time pulsatility signal 4 have been measured.

In yet another embodiment, the calculating module 52 can be configured for calculating the BP value of a user by further using the user-specific information. This step is also known as a calibration step, an initialization step or a re-initialization step.

In yet another embodiment, the calculating module 52 can be further configured for calculating the BP value of a user by further using the triggering input.

The calculating module 52 can be configured for calculating the BP value by using on a pulse wave analysis technique, for example such as the pulse wave analysis technique described in European patent application EP3226758.

The calculating module 52 can be further be configured for calculating the BP value by using on a machine learning technique, for example such as described in Fen Miao et. al., “A Novel Continuous Blood Pressure Estimation Approach Based on Data Mining Techniques”, IEEE Journal of Biomedical and Health Informatics, Volume: 21, Issue: 6, pp. 1730-1740, 2017.

In a further embodiment, the calculating module 52 can be configured for calculating BP related physiological parameters from the pulsatility signals including any one of: systolic BP, diastolic BP or mean arterial pressure.

The calculating module 52 can be further configured for calculating additional physiological parameters including any one of: pulse pressure, central pulse wave velocity, peripheral pulse wave velocity, arterial stiffness, aortic pulse transit time, augmentation index, stroke volume, stroke volume variations, pulse pressure variations, cardiac output, systemic vascular resistance, venous pressure, systemic hemodynamic parameters, pulmonary hemodynamic parameters, cerebral hemodynamic parameters, heart rate, heart rate variability, inter-beat intervals, arrhythmias detection, ejection duration, SpO₂, SpHb, SpMet, SpCO, respiratory rate, tidal volume, apnea detection, sleep quality, sleep scoring, sleep analysis, bed time, sleep duration, rem sleep time, light sleep time, deep sleep time, time to get up, time to sleep, sleep efficiency, minutes awake after sleep onset, snoring duration, stress indexes, and general cardiovascular and health indexes.

In another embodiment, the database storage system 51 can be configured to store the calculated BP values (represented by the symbol “BPi” with i=1 to n) and/or the additional physiological parameters in the database 53.

Display

In an embodiment, the system can comprise a display interface 8 for displaying the calculated BP values and/or additional physiological parameters stored in the database 53.

The display interface 8 can be comprised in the external service module 5 (see FIG. 5). The display interface 8 can comprise a web interface or similar connected software that can safely communicate with the external service module 5 (not shown). A clinician and/or user may use a webpage of the web interface to connect to the external service module and see the data comprised in the database 53. Alternatively, the display interface 8 can also be comprised in an application running on the portable gateway device 30 (such as a smartphone).

The display interface 8 can be comprised in the wearable device 1. The external service module 5 can then be configured for transmitting the calculated BP value and/or additional physiological parameters to the wearable device 1 such that the latter can be displayed on the display interface 8.

The display interface 8 can be comprised in the portable gateway device 3. The external service module 5 can then be configured for transmitting the calculated BP value and/or additional physiological parameters to the portable gateway device 3 such that the latter can be displayed on the display interface 8.

The external service module 5 can be further configured for transmitting the calculated BP values to the portable gateway device 3. The transmitted calculated BP values can then be displayed on the portable gateway device 3.

### REFERENCE NUMBERS

1 wearable device
11 pulsatility sensing unit
110 wrist's skin
111 ulnar artery
112 radius artery
113 ulna bone
114 radius bone
115 light source
116 photodetector
117 capillary bed
12 motion sensor
13 geolocation means
15 armband
2 signal module
20 controlling module
21 processing module
22 pulsatility signal data
23 triggering module
24 first short range data buffer
25 first short range communication link
26 long range data buffer
27 second short range communication link
28 second short range data buffer
3 communication module
30 portable gateway device
4 pulsatility signal
5 external service module
51 database storage system
52 calculating module
53 database
6 independent BP measurement
7 long range communication link
8 display interface

1. System for determining a blood pressure (BP) of one or a plurality of users, the system comprising,
for each user, a signal module configured to cooperate with a wearable device destined to be worn on a wrist of the user and comprising a pulsatility sensing unit; the signal module comprising a controlling module configured to control the pulsatility sensing unit such that the pulsatility sensing unit measures a plurality of pulsatility signals at the user's wrist; a processing module configured for processing the pulsatility signals to obtain pulsatility signal data; the signal module further comprising a communication module for remotely transmitting said pulsatility signal data;
the system further comprising an external service module including a database storage system for storing in a database the transmitted pulsatility signal data for each of said one or a plurality of users; and a calculating module configured for calculating a BP value for each of said one or a plurality of users, based on the pulsatility signal data stored in the database;
wherein the database storage system is configured to store a set comprising a plurality of pulsatility signal data obtained for the wearable device of each user; and
wherein the calculating module is further configured for calculating the BP based on a set comprising a plurality of pulsatility signal data stored in the database for each of said one or a plurality of user.

2. The system according to claim 1,
wherein the pulsatility sensing unit comprises an optical measuring sensor.

3-10. (canceled)

11. The system according to claim 1,
wherein the controlling module is configured such that each of said plurality of pulsatility signals is measured during a predetermined measurement time period; wherein the controlling module comprises a triggering module configured to initiate and end said measurement time period;
and
wherein the triggering module is configured to control the pulsatility sensing unit according to a trigger parameter or to a triggering input entered manually.

12-13. (canceled)

14. The system according to claim 11,
wherein the wearable device further comprises a motion sensor measuring a motion signal corresponding to a user's motion when the wearable device is worn,
the trigger parameter comprising the motion signal; or
wherein the trigger parameter comprises a user-related behavioral information.

15-17. (canceled)

18. The system according to claim 11,
wherein the communication module is configured for transmitting said trigger parameter or said triggering input to the external service module.

19-21. (canceled)

22. The system according to claim 1,
wherein the processing module is configured to perform a pre-processing step on the measured pulsatility signals such as to obtain said pulsatility signal data; and
wherein said pre-processing step comprises any one of: a lossless compression of the measured pulsatility signals, or executing an ensemble averaging algorithm on the measured pulsatility signal.

23-27. (canceled)

28. The system according to claim 1,
wherein the signal module is remote from the wearable device and the signal module cooperates with the wearable device via a first short range communication link; and
wherein the signal module comprises a first short range data buffer adapted to store the measured pulsatility signals when they are not transmitted by first short range communication link; and/or
wherein the wearable device comprises a long range data buffer adapted to store the pulsatility signal data when they are not transmitted by communication module.

29. (canceled)

30. The system according to claim 1,
wherein the communication module is comprised in a portable gateway device configured for communicating with the signal module and the external service module.

31-36. (canceled)

37. The system according to claim 1,
wherein the database storage system is configured for storing in the database any one of:
the pulsatility signal data for each user, the trigger parameter for each pulsatility signal data, or the triggering input for each pulsatility signal data.

38-40. (canceled)

41. The system according to claim 1,
configured for inputting a user-specific information for each of said one or a plurality of users.

42-43. (canceled)

44. The system according to claim 1,
wherein said user-specific information comprises one or a plurality of reference BP measurements, each measured independently from to the measurement performed with the pulsatility sensing unit;
or
wherein said one or a plurality of reference BP measurements is measured simultaneously with the measurement performed with the pulsatility sensing unit.

45-46. (canceled)

47. The system according to claim 1,
wherein the database storage system is configured for storing said user-specific information in the database.

48. The system according to claim 1,
wherein the portable gateway device is configured for introducing said user-specific information in the database.

49. The system according to claim 1,
wherein the calculating module configured for calculating the BP value of a user, based on any one of:
the pulsatility signal data stored in the database; or
a set comprising a plurality of pulsatility signal data stored in the database for the user, or
a subset of said set comprising one or a plurality of pulsatility signal data; or
the pulsatility signal data stored in the database for a subset of users.

50-53. (canceled)

54. The system according to claim 1,
wherein the calculating module is configured for calculating the BP value of a user by further using said user-specific information and/or said triggering input.

55-57. (canceled)

58. The system according to claim 1,
wherein the calculating module is further configured for calculating other physiological parameters including any one of: systolic BP, diastolic BP or mean arterial pressure, pulse pressure, central pulse wave velocity, peripheral pulse wave velocity, arterial stiffness, aortic pulse transit time, augmentation index, stroke volume, stroke volume variations, pulse pressure variations, cardiac output, systemic vascular resistance, venous pressure, systeric hemodynamic parameters pulmonary hemodynamic parameters, cerebral hemodynamic parameters heart rate, heart rate variability, inter-beat intervals, arrhythmias detection, ejection duration, SpO₂, SpHb, SpMet, SpCO, respiratory rate, tidal volume, apnea detection, sleep quality, sleep scoring, sleep analysis, bed time, sleep duration, rem sleep time, light sleep time, deep sleep time, time to get up, time to sleep, sleep efficiency, minutes awake after sleep onset, snoring duration, stress indexes, and general cardiovascular or health indexes.

59. (canceled)

60. The system according to claim 1,
comprising a display interface configured for displaying the calculated BP value.

61-64. (canceled)

65. The system according to claim 1,
wherein the calculating module is configured for calculating the BP value of one or a plurality of users, based on the pulsatility signal data, or the trigger parameter for each pulsatility signal data, or the triggering input for each pulsatility signal data, or the user-specific, information, stored in the database from a plurality of users;
wherein the calculating module is configured for calculating the BP and for training the calculating technique.
